(19) United States
(12) Patent Application Publication
(10) Pub. No.: US 2017/0144042 A1
(43) Pub. Date: May 25, 2017

(54) MOBILE TERMINAL, TRAINING MANAGEMENT PROGRAM AND TRAINING MANAGEMENT METHOD

(71) Applicant: KYOCERA CORPORATION, Kyoto (JP)

(72) Inventor: Atsuhiko KANDA, Yokohama-shi (JP)

(21) Appl. No.: 15/416,836

(22) Filed: Jan. 26, 2017

Related U.S. Application Data
(63) Continuation of application No. PCT/JP2015/069509, filed on Jul. 7, 2015.

(30) Foreign Application Priority Data
Jul. 29, 2014 (JP) 2014-154018

(51) Int. Cl. A63B 69/00; A61B 5/00; A61B 5/024; A63B 71/06; A63B 24/00

(52) U.S. Cl. CPC A63B 69/00; A63B 71/06; A63B 24/0062; A61B 5/024; A61B 5/7203; A61B 5/6803; A61B 5/002; A61B 5/742; A61B 5/7475; A61B 5/7225; A63B 2220/34; A63B 2220/40; A63B 2220/74; A63B 2230/06; A63B 2225/50

(57) ABSTRACT

A mobile terminal includes a memory and at least one processor. The memory stores details of a training having been performed and a heart rate in the training in association with each other. The processor obtains a heart rate of the user, an attitude of the mobile terminal and an atmospheric pressure surrounding the mobile terminal. While a first training is being performed, the processor detects details of the first training from the attitude and the atmospheric pressure. The processor determines whether a training with details corresponding with the details of the first training has been performed. When the details of the first training correspond with details of a second training having been performed, the corrects the maximum heart rate based on a first heart rate associated with the first training and a second heart rate associated with the second training.

### CROSS-REFERENCE TO RELATED APPLICATION

The present application is a continuation based on PCT Application No. PCT/JP2015/069509 filed on Jul. 7, 2015, which claims the benefit of Japanese Application No. 2014-154018 filed on Jul. 29, 2014. PCT Application No. PCT/JP2015/069509 is entitled “Portable Terminal, Training Management Program, and Training Management Method”, and Japanese Application No. 2014-154018 is entitled “Mobile Terminal, Training Management Program and Training Management Method.” The content of which are incorporated by reference herein in their entirety.

### FIELD

Embodiments of the present disclosure relate to a mobile terminal, a training management program and a training management method, and more particularly to a mobile terminal possessed by a user performing a training, a training management program and a training management method.

### BACKGROUND

A calorie measuring device is known. The calorie measuring device may be included in a smartphone, for example. The sex, age, height, weight, and the like of a user are input to the calorie measuring device, and a normal heart rate, a resting heart rate and the like of the user are measured in advance. The user can measure consumed calories by measuring his/her heart rate before and after a training.

### SUMMARY

An aspect is a mobile terminal for managing a training performed by a user based on a predetermined maximum heart rate. The mobile terminal includes a memory and at least one processor. The memory is configured to store details of a training having been performed and a heart rate in the training in association with each other. The at least one processor is configured to obtain a heart rate of the user, an attitude of the mobile terminal and an atmospheric pressure surrounding the mobile terminal. While a first training is being performed, the at least one processor is configured to detect details of the first training from the attitude and the atmospheric pressure. The at least one processor is configured to determine whether a training with details corresponding with the details of the first training has been performed. When the details of the first training correspond with details of a second training having been performed, the at least one processor is configured to correct the maximum heart rate based on a first heart rate associated with the first training and a second heart rate associated with the second training.

Another aspect is a processor-readable non-transitory recording medium with a program recorded thereon. The program causes a processor of a mobile terminal to execute a training management method for managing a training performed by a user based on a predetermined maximum heart rate. The training management method includes obtaining a heart rate of the user, an attitude of the mobile terminal and an atmospheric pressure surrounding the mobile terminal. The training management method includes, while a first training is being performed, detecting details of the first training from the attitude and the atmospheric pressure. The training management method includes determining whether a training with details corresponding with the details of the first training has been performed. The training management method includes, when the details of the first training correspond with details of a second training having been performed, correcting the maximum heart rate based on a first heart rate associated with the first training and a second heart rate associated with the second training.

Still another aspect is a training management method for managing a training performed by a user based on a predetermined maximum heart rate. The training management method is executed by a processor of a mobile terminal. The training management method includes obtaining a heart rate of the user, an attitude of the mobile terminal and an atmospheric pressure surrounding the mobile terminal. The training management method includes, while a first training is being performed, detecting details of the first training from the attitude and the atmospheric pressure. The training management method includes determining whether a training with details corresponding with the details of the first training has been performed. The training management method includes, when the details of the first training correspond with details of a second training having been performed, correcting the maximum heart rate based on a first heart rate associated with the first training and a second heart rate associated with the second training.

The foregoing and other objects, features, aspects and advantages of the present disclosure will become more apparent from the following detailed description of the present disclosure when taken in conjunction with the accompanying drawings.

### BRIEF DESCRIPTION OF THE DRAWINGS

FIG. 1 is an illustration showing an example of a state where a mobile phone and a headphone of one embodiment of the present disclosure are used.

FIG. 2 is an outline view showing an example of the appearance of the mobile phone shown in FIG. 1.

FIG. 3 is an illustration showing an example of an electric configuration of the mobile phone shown in FIG. 1.

FIG. 4 is an outline view showing an example of the appearance of the headphone shown in FIG. 1.

FIG. 5 is an illustration showing an example of an electric configuration of the headphone shown in FIG. 1.

FIG. 6 is an illustration showing an example of a configuration of data indicating a heart rate for each exercise intensity based on the maximum heart rate (hereinafter briefly referred to as maximum-heart-rate data) stored in a RAM shown in FIG. 3.

FIG. 7 is an illustration showing an example of a memory map of the RAM of the mobile phone shown in FIG. 3.

FIG. 8 is a flowchart showing an example of a training management process executed by a processor shown in FIG. 3.

### DETAILED DESCRIPTION

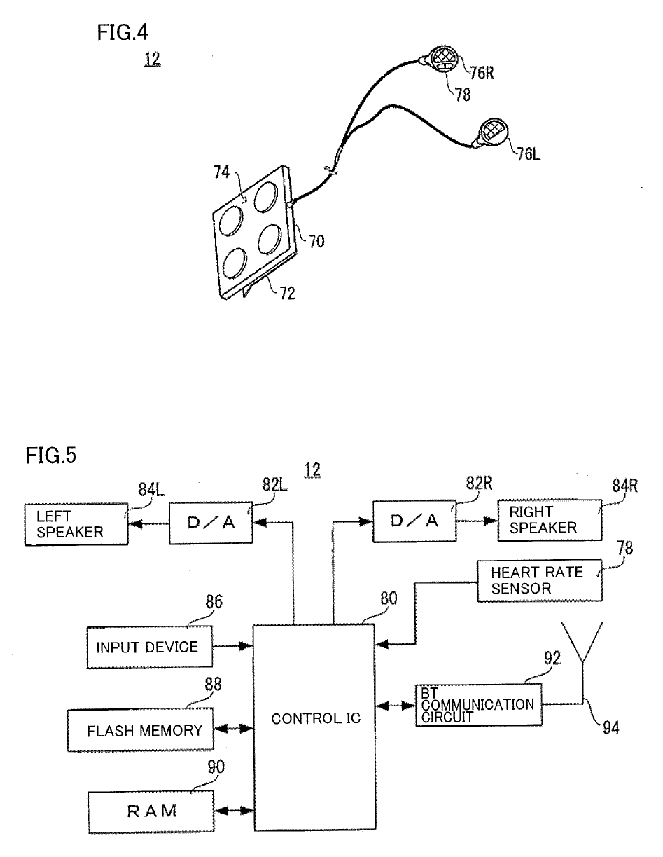

Referring to FIG. 1, a mobile phone 10 of one embodiment of the present disclosure is a smartphone as an example, and is possessed by a user. The user can wear a headphone 12 on his/her ear, and can fix a housing 70 (see FIG. 4) of headphone 12 to his/her clothes. Mobile phone 10 may be wirelessly connected to headphone 12 using a Bluetooth® wireless communication technique.

Mobile phone 10 may have a music player function. When an operation for playing back music on mobile phone 10 is performed, music may be output from headphone 12 wirelessly connected thereto. As will be described later in detail, headphone 12 can detect the heart rate of a user, and can transmit the detected heart rate to mobile phone 10.

It is pointed out in advance that the present disclosure is applicable to any mobile terminal, such as a tablet terminal, a tablet type PC (Personal Computer) and a PDA (Personal Data Assistant).

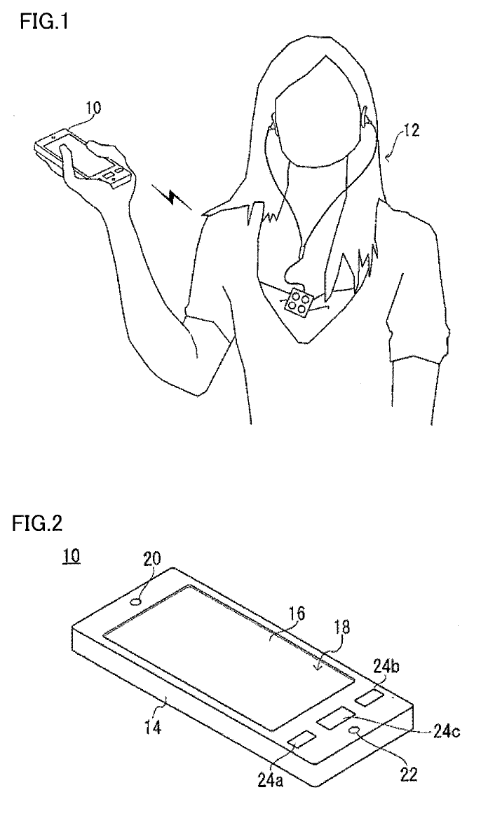

Referring to FIG. 2, mobile phone 10 can include a vertically-long flat rectangular housing 14. A display 16 may be located on a major surface (front surface) of housing 14. Display 16 is implemented by a liquid crystal display, an organic EL (Electro Luminescence) display or the like, for example. A touch panel 18 may be located on display 16.

A speaker 20 may be built in longitudinal one end of housing 14 on the major surface side, and a microphone 22 may be built in the longitudinal other end on the major surface side.

In an embodiment, a talk key 24a, a call release key 24b and a menu key 24c may be located on the major surface of housing 14 as hard keys 24 constituting input operation means together with touch panel 18.

For example, a user can start a voice call by performing a touch operation on a dial pad displayed on display 16 to input a telephone number and then operating talk key 24a. The user can terminate a voice call by operating call release key 24b. The user can turn on or off mobile phone 10 by pressing and holding call release key 24b. The user can turn off display 16 and touch panel 18 by briefly pressing call release key 24b with the screen being displayed on display 16.

The user can cause a home screen to be displayed on display 16 by operating menu key 24c. The user can perform a touch operation via touch panel 18 on a GUI (Graphical User Interface) being displayed on display 16 in that state to select an object and can make the selection settled.

In the following description, GUIs, such as icons and soft keys displayed on display 16, may be collectively called an object.

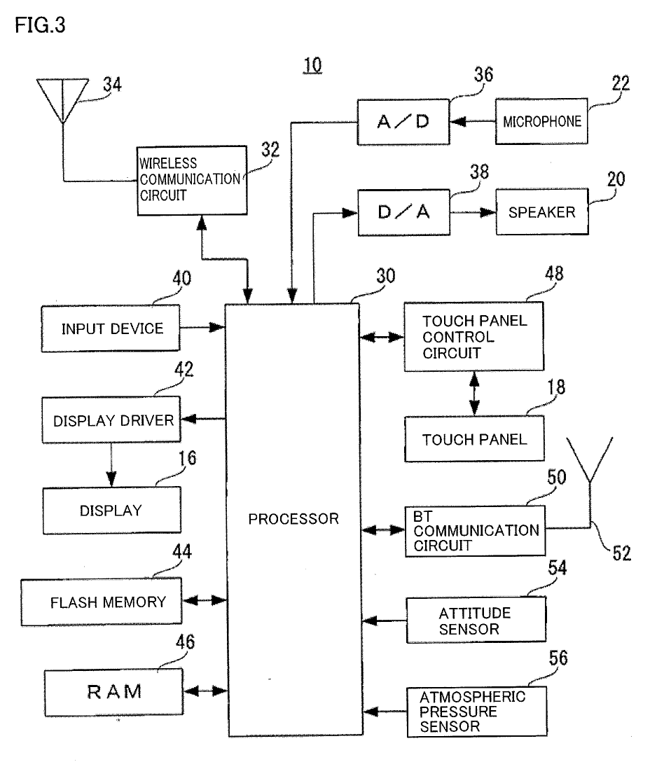

Referring to FIG. 3, mobile phone 10 of the embodiment shown in FIG. 2 includes at least one processor 30 for providing control and processing capability to perform various functions as described in further detail below. In accordance with various embodiments, at least one processor 30 may be implemented as a single integrated circuit (IC) or as multiple communicatively coupled ICs and/or discrete circuits. It is appreciated that at least one processor 30 can be implemented in accordance with various known technologies. In one embodiment, the processor includes one or more circuits or units configurable to perform one or more data computing procedures or processes by executing instructions stored in an associated memory, for example, In other embodiments, at least one processor 30 may be implemented as firmware (e.g. discrete logic components) configured to perform one or more data computing procedures or processes. In accordance with various embodiments, at least one processor 30 may include one or more processors, controllers, microprocessors, microcontrollers, application specific integrated circuits (ASICs), digital signal processors, programmable logic devices, field programmable gate arrays, or any combination of these devices or structures, or other known devices and structures, to perform the functions described herein. At least one processor 30 includes a CPU (Central Processing Unit) and may be called a computer, for example. At least one processor 30 includes a storage element. The storage element is a SRAM (Static Random Access Memory) or a DRAM (Dynamic Random Access Memory), for example.

A wireless communication circuit 32, an A/D (Analog/Digital) converter 36, a D/A (Digital/Analog) converter 38, an input device 40, a display driver 42, a flash memory 44, a RAM (Random Access Memory) 46, a touch panel control circuit 48, a Bluetooth® (hereinafter referred to as BT) communication circuit 50, an attitude sensor 54, an atmospheric pressure sensor 56, and the like may be connected to processor 30. An antenna 34 may be connected to wireless communication circuit 32. Microphone 22 may be connected to A/D converter 36, Speaker 20 may be connected to D/A converter 38. Display 16 may be connected to display driver 42. Touch panel 18 may be connected to touch panel control circuit 48. A BT antenna 52 may be connected to BT communication circuit 50.

Processor 30 manages overall control of mobile phone 10. All or part of a program preset in flash memory 44 is developed to RAM 46 when in use. Processor 30 can operate in accordance with the program on this RAM 46. RAM 46 can be used as a working area or a buffer area of processor 30.

Input device 40 includes three hard keys 24 shown in FIG. 2, Input device 40 can receive a key operation on each of hard keys 24. Information (key data) on each of hard keys 24 for which a key operation has been received may be input to processor 30 by input device 40. In various embodiments, input device 40 may be implemented using any input technology or device known in the art such as, for example, a QWERTY keyboard, a pointing device (e.g., a mouse), a joy stick, a stylus, a touch screen display panel, a key pad, one or more buttons, etc., or any combination of these technologies.

Wireless communication circuit 32 includes a circuit for transmitting and receiving electric waves for a voice call or e-mail via antenna 34. In an embodiment, wireless communication circuit 32 includes a circuit for making wireless communications by the CDMA (Code Division Multiple Access) technology. For example, based on an operation for call origination (voice transmission) received by touch panel 18, wireless communication circuit 32 can execute a voice transmission process under an instruction from processor 30, and can output a voice transmission signal via antenna 34. The voice transmission signal may be transmitted to the other party's phone through a base station and a communication network. When a voice arrival process is performed in the other party's phone, a state where a communication can be made is established, and processor 30 can execute a calling process.

A/D converter 36 can convert an analog audio signal obtained from microphone 22 into digital audio data as described above, and can input the audio data to processor 30. D/A converter 38 can convert digital audio data into an analog audio signal, and can supply the signal to speaker 20 via an amplifier. Voice based on the audio data may be output from speaker 20. With a calling process being executed, voice collected by microphone 22 may be transmitted to the other party's phone, and voice collected at the other party's phone may be output from speaker 20.

Display 16 shown in FIG. 2 is connected to display driver 42. Display 16 can display video or an image in accordance with video data or image data output from processor 30. Display driver 42 includes a video memory for temporarily storing data to be displayed. Data output from processor 30 may be stored in this video memory. Display driver 42 can cause display 16 to display an image in accordance with the contents of the video memory. Display driver 42 can control display 16 connected to display driver 42 under an instruction from processor 30. Display 16 may include a backlight. Display driver 42 can control the brightness and turning on and off of the backlight in accordance with instructions from processor 30.

Touch panel 18 shown in FIG. 2 may be connected to touch panel control circuit 48, Touch panel control circuit 48 can apply a voltage or the like necessary for touch panel 18. Touch panel control circuit 48 can input, to processor 30, a touch start signal indicating the start of a touch on touch panel 18, an end signal indicating the end of a touch, and coordinate data indicating a touched position. Processor 30 can determine a touched object based on this coordinate data and a change in that coordinate data.

For example, when touch panel 18 is touched, a touched region may be detected by touch panel 18. Touch panel control circuit 48 can regard the center of gravity of the touched region as a touched position, and can input coordinates of the center of gravity to processor 30. The center of gravity of the touched region in a touch operation may indicate a touch start position, an end position or a currently-touched position. In another embodiment, a position at which a finger or the like initially touches touch panel 18 rather than the center of gravity may be regarded a touched position.

Touch panel 18 includes a capacitive touch panel for detecting a change in capacitance occurring between its surface and an object such as a finger (hereinafter comprehensively referred to as a finger for convenience). Touch panel 18 can detect that one or several fingers, for example, have touched touch panel 18. A user can input an operated position, an operation direction and the like to mobile phone 10 by performing a touch operation on the surface of touch panel 18. Touch panel 18 may be called a pointing device.

A touch operation of an embodiment includes a tap operation, a long tap operation, a flick operation, a swipe (sliding) operation, and the like.

The tap operation is an operation of contacting (touching) the surface of touch panel 18 with a finger, and then lifting (releasing) the finger from the surface of touch panel 18 within a short period of time. The long tap operation is an operation of continuously contacting the surface of touch panel 18 with a finger for a predetermined time or longer, and then lifting the finger from the surface of touch panel 18. The flick operation is an operation of contacting the surface of touch panel 18 with a finger, and flicking the finger in any direction at a predetermined speed or higher. The swipe (sliding) operation is an operation of moving a finger in any direction with the finger kept in contact with the surface of touch panel 18, and then lifting the finger from the surface of touch panel 18.

The above-mentioned swipe operation also includes a drag operation, which is a swipe operation of contacting an object displayed on the surface of display 16 with a finger and then moving the object. An operation of lifting the finger from the surface of touch panel 18 after a drag operation will be called a drop operation.

In the following description, a tap operation, a long tap operation, a flick operation, a swipe operation, a drag operation, and a drop operation may each be described with the word “operation” omitted therefrom. A touch operation may be performed not only with a user's finger but also with a stylus pen or the like.

In an embodiment, when a predetermined time (e.g., 15 seconds) has passed with no operation performed, display 16 and touch panel 18 may be turned off automatically.

BT communication circuit 50 can establish a BT wireless communication in which a master-slave relationship is built between BT communication circuit 50 and another communication device, such as headphone 12 or a headset.

For example, when a BT communication is enabled in mobile phone 10, and mobile phone 10 is set to operate as a master, mobile phone 10 can search for a communication device which operates as a slave. When a user performs an operation of making a BT wireless communication with headphone 12, headphone 12 of an embodiment can operate as a slave to transition to a “connection standby state” responsive to a connection request of a master terminal. Mobile phone 10 operating as a master can find headphone 12 operating as a slave. When mobile phone 10 finds headphone 12, a user may be requested to input a pass code (PIN: Personal Identification Number) set for headphone 12. When mobile phone 10 receives a proper PIN input, a BT communication may be established between mobile phone 10 and headphone 12. When a BT communication is established, data transmission and reception can be performed. In an embodiment, music data or the like may be transmitted from mobile phone 10 to headphone 12, and data on an operation for the music player function, data indicating a heart rate or the like may be transmitted from headphone 12 to mobile phone 10.

Attitude sensor 54 detects an inclination and a movement of mobile phone 10. For example, attitude sensor 54 can include a gyro sensor for detecting rotations (angular velocity) of mobile phone 10 about three axes (X, Y, Z), and an acceleration sensor for detecting accelerations of mobile phone 10 in the three axial directions (X, Y, Z), the gyro sensor and the acceleration sensor being integral with each other by a MEMS (Micro Electro Mechanical Systems) technique. Attitude sensor 54 may also be called a six-axis motion sensor. Processor 30 can detect an inclination (angle) and a movement of mobile phone 10 based on triaxial angular velocities and triaxial accelerations that can be output from attitude sensor 54.

For example, when a certain screen is displayed on display 16, processor 30 can detect the attitude in which mobile phone 10 is held using the angular velocities and accelerations. A display direction may be set in accordance with the detected attitude. In an embodiment, when mobile phone 10 is held in the vertical attitude, the display direction is set at the portrait orientation, and when mobile phone 10 is held in the lateral attitude, the display direction may be set at the landscape orientation.

In another embodiment, the acceleration sensor and the gyro sensor may each be used instead of attitude sensor 54.

Atmospheric pressure sensor 56 is a semiconductor pressure sensor, and can detect a surrounding atmospheric pressure using a piezoresistive element located therein. Processor 30 can convert an output of atmospheric pressure sensor 56 into an atmospheric pressure value and calculate a pressure altitude based on the atmospheric pressure value. For example, in an embodiment, the inclination of a road on which a user is moving may be estimated using the calculated pressure altitude.

In another embodiment, a capacitive atmospheric pressure sensor in accordance with the MEMS technique or the like may be used.

In another embodiment, attitude sensor 54 and atmospheric pressure sensor 56 may be integral with each other.

Referring to FIG. 4, headphone 12 includes flat rectangular housing 70. A clip 72 for fixing housing 70 to clothes or the like may be located on the back surface of housing 70. An operation key group 74 for operating headphone 12 may be located on the front surface of housing 70. A cable bifurcated partway may be connected to a side surface of housing 70. A right-ear mounted unit 76R and a left-ear mounted unit 76L may be connected to the leading ends of the cable, respectively. The two ear mounted units have an in-ear type shape, and may be worn on the ears of a user, respectively. A right speaker 84R (see FIG. 5) and a heart rate sensor 78 are located in right-ear mounted unit 76R, and a left speaker 84L (see FIG. 5) is located in left-ear mounted unit 76L.

The term “unit” as used herein refers to known structures such as hardware, firmware, non-transitory computer-readable media that stores computer-executable instructions, or any combination of these elements, for performing the associated functions described herein. Additionally, various units can be discrete units; however, as would be apparent to one of ordinary skill in the art, two or more units may be combined to form a single unit that performs the associated functions according to various embodiments of the disclosure. Conversely, a single unit may be divided into two or more units that perform respective associated functions according to various embodiments of the disclosure.

For example, a user can fix headphone 12 (housing 70) to his/her own clothes or the like using clip 72. The user can operate each operation key included in operation key group 74 to turn on or off headphone 12 or to cause headphone 12 to transition to the “connection standby state” in a BT communication. With mobile phone 10 and headphone 12 making a BT communication, the user can perform an operation, such as music playback, stop or fast forward, by operation key group 74. The user can measure his/her heart rate during a training by headphone 12. The user can also check the measured heart rate on mobile phone 10, The user wearing headphone 12 can easily measure his/her own heart rate.

Referring to FIG. 5, headphone 12 of the embodiment shown in FIG. 1 includes a control IC (Integrated Circuit) 80 also called a computer or a CPU. Heart rate sensor 78, a D/A converter 82L, a D/A converter 82R, an input device 86, a flash memory 88, a RAM 90, a BT communication circuit 92, and the like may be connected to control IC 80, Left speaker 84L may be connected to D/A converter 82L, right speaker 84R may be connected to D/A converter 82R, and a BT antenna 94 may be connected to BT communication circuit 92.

Control IC 80 can manage overall control of headphone 12. All or part of a program preset in flash memory 88 may be developed to RAM 90 when in use. Control IC 80 can operate in accordance with the program on RAM 90,

Input device 86 includes each operation key of operation key group 74 shown in FIG. 4. When each operation key receives a key operation, information on the operation key may be input to control IC 80 by input device 86.

D/A converter 82L and D/A converter 82R can convert digital audio data received by headphone 12 into an analog audio signal, and can supply the signal to left speaker 84L and right speaker 84R via an amplifier. When the audio data is applicable to stereo playback, control IC 80 can output audio data of channels corresponding to D/A converter 82L and D/A converter 82R, respectively. Sound of different channels may be output from left speaker 84L and right speaker 84R, respectively.

BT communication circuit 92 can establish a BT wireless communication in which a master-slave relationship is built between BT communication circuit 92 and another communication device, such as mobile phone 10, similarly to BT communication circuit 50. For example, when an operation key is operated to enable a BT communication, BT communication circuit 92 can operate as a slave, and can transition to the “connection standby state.” Since establishment of a BT communication with mobile phone 10 has been described above, detailed description thereof will not be repeated.

Heart rate sensor 78 can include a LED (Light Emitting Diode) which emits red light and a phototransistor. When heart rate sensor 78 operates, the LED emits red light on the surface of an ear on which it is mounted, and a pulse including changes in blood passing through a blood vessel within the ear may be captured by the phototransistor. In an embodiment, the pulse rate measured on the user's ear may be transmitted to mobile phone 10 as the heart rate of a user. Heart rate sensor 78 may also be called a heart-rate measuring sensor.

Mobile phone 10 of an embodiment may have a training controlling function. This training controlling function can calculate calories consumed by a training performed by a user (hereinafter referred to as consumed calories), and can obtain exercise intensity when that training is performed. The training controlling function can manage a training performed by the user by obtaining consumed calories and exercise intensity when the user performs the training.

When the training controlling function is executed initially, the user can register user information, such as his/her height, weight, age, and sex. When the age is registered as user information, the maximum heart rate based on the age may be calculated, and the maximum heart rate may also be registered as one piece of user information. A heart rate measured with predetermined conditions being satisfied may be regarded as a resting heart rate, and the resting heart rate may also be registered as one piece of user information.

The maximum heart rate may be calculated further using an additional numerical value (e.g., maximum oxygen uptake or the like). Since a method of calculating the maximum heart rate based on the age is widely known in common, detailed description thereof will not be given.

When the user information is registered, the exercise intensity of the training performed by the user can be obtained. In an embodiment, the exercise intensity may be obtained in accordance with the Karvonen Formula from the heart rate during the training, the resting heart rate and the maximum heart rate. In another embodiment, the exercise intensity may be obtained using another numerical value.

For example, the exercise intensity may differ between a training for burning fat and a training for increasing the cardiopulmonary function. When the user presets an effect to be obtained by a training, the training controlling function can measure the heart rate of the user performing the training, and can manage whether during the training a necessary exercise intensity has been obtained. The training controlling function can also recommend a training suitable for the user.

In an embodiment, consumed calories of a training performed by the user can also be calculated. In an embodiment, a maximum-heart-rate table in which a current heart rate relative to the maximum heart rate and consumed calories per unit time (e.g., 1 minute) are associated with each other may be created in advance, and consumed calories when the training is performed may be obtained using the maximum-heart-rate table.

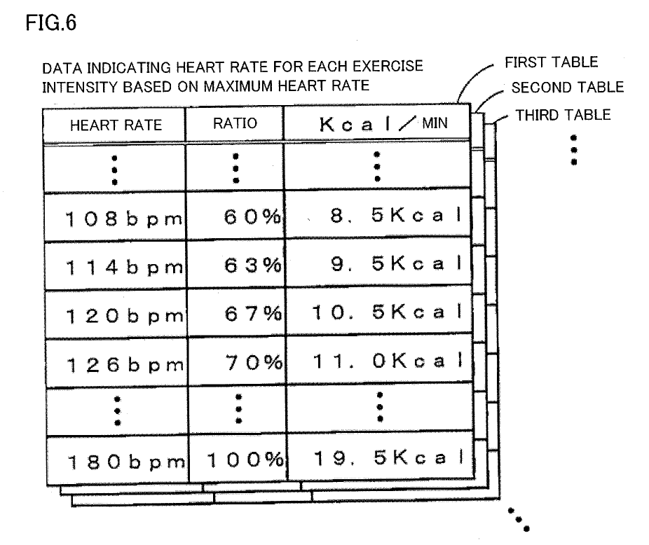

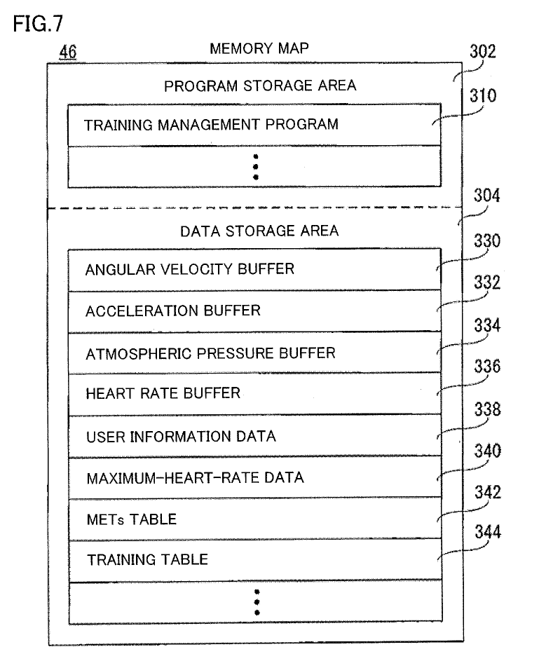

FIG. 6 shows an example of maximum-heart-rate data 340 (see a memory map of FIG. 7) including a plurality of tables. For example, when the heart rate is 108 bpm (60%) in a first table in which the maximum heart rate is set at 180 bpm, consumed calories per unit time will be 8.5 Kcal. Consumed calories per unit time will be 9.5 Kcal when the heart rate is 114 bpm (63%). Consumed calories per unit time will be 10.5 Kcal when the heart rate is 120 bpm (67%), and consumed calories per unit time will be 11.0 Kcal when the heart rate is 126 bpm (70%). Consumed calories per unit time will be 19.5 Kcal when the heart rate is 180 bpm (100%). The maximum heart rate, the value of consumed calories per unit time and the like may differ between the first table and a third table in an embodiment.

The training controlling function can set a maximum-heart-rate table suitable for the user among these maximum-heart-rate tables based on registered user information, such as the maximum heart rate, height, weight, age, and sex. When the user performs a training, consumed calories may be obtained based on the heart rate measured during the training.

The training controlling function can detect the details of a training being performed by the user based on the accelerations and angular velocities detected by attitude sensor 54 as well as the pressure altitude calculated based on the output of atmospheric pressure sensor 56. The type of a movement being made by the user can be estimated from how the accelerations change. The type of a training performed by the user (e.g., walking, running, cycling, or the like) can be estimated from the accelerations. The speed at which the user is moving can be estimated from the accelerations and angular velocities. The inclination of a road on which the user is moving (including stairs and the like) can be estimated from the pressure altitude. By combining these estimations, an analysis result of the training performed by the user, such as “running at 9.7 km/h on a road with an inclination of 5%”, may be obtained. The details of the training can be analyzed using a sensor commonly included in mobile phone 10. In an embodiment, the analysis result of the details of the training may be used as a detection result.

In another embodiment, when a training to be started from now on is set by a user's operation or the like, the set result may be used as a detection result.

When the details of the training are detected, the training controlling function can associate the heart rate during the training with the details of the training to obtain training information, and can register the training information in a training table 344 (see the memory map of FIG. 7). A history of trainings performed by the user may be registered in training table 344. When an operation of checking the history of trainings is performed, the history of trainings may be displayed on display 16. The user can freely check the history of trainings he/she has performed. By checking the history of trainings, the user can make future training plans.

The training controlling function can calculate METs (“Metabolic Equivalent of Task” or “Metabolic equivalents”) from the speed at which the user is moving and the inclination of the road on which the user is moving. METs indicates how much calories are consumed when one performs an activity or movement relative to the resting state. For example, “1 METs” is obtained when one is reading a book or the like while sitting down. In an embodiment, METs may be obtained using a METs table 342 (see the memory map of FIG. 7) created by the inventor based on results obtained through experiment. For example, in METs table 342, the horizontal axis indicates the speed and the vertical axis indicates the inclination. METs may be obtained from the speed at which the user is moving and the inclination of the road on which the user is moving. Since the usage of METs will be described later, detailed description thereof will not be given here.

When the user continuously performs a training, the user's cardiopulmonary function may be improved. As the cardiopulmonary function is improved, the heart rate may be lowered even when a training with the same details is performed, and the exercise intensity when the training is performed may also be lowered. If the exercise intensity when the same training is performed is lowered, consumed calories measured when the training is performed may not present a correct value. It can be said that the state where consumed calories are not measured appropriately is a state where the training performed by the user is not managed appropriately. When the user continuously performs a training with the same details, an effect that the user expects may not be obtained.

An embodiment allows a training performed by the user to be managed appropriately by correcting the registered maximum heart rate of the user. When the user performs a training, it may be determined whether the same training as the training being currently performed has been performed from the registered history of trainings. When it is determined that the same training has been performed, the difference (or also called a heart-rate relation value) between a past heart rate when the training was performed and a current heart rate while the training is being performed may be calculated. It may be determined whether the cardiopulmonary function has been changed and the exercise intensity has been changed. When the difference in heart rate is larger than a threshold value (e.g., 5 bpm), a correction value for correcting the maximum heart rate may be calculated.

The correction value may be calculated by estimating a difference between the exercise intensity obtained by a training in the past and the exercise intensity obtained by a current training. In an embodiment, METs may be used for estimating the difference in exercise intensity. For example, when the user's cardiopulmonary function has been improved, the heart rate was raised in the past with a training of 5 METs, while the heart rate may not be raised currently as before even with a training of 5 METs. If METs of a training which will raise the heart rate as before is found, the difference in exercise intensity can be estimated from two METs.

In a specific procedure for obtaining METs, consumed calories per unit time (hereinafter “first consumed calories”) may be obtained based on the heart rate when the training was performed in the past from the maximum-heart-rate table having been set.

Calories per unit time to be consumed by the current training (hereinafter “second consumed calories”) may be obtained. In an embodiment, the current METs is read from METs table 342 described above, and the current consumed calories may be calculated based on Expression (1) indicated below. The registered weight of the user is substituted for “weight” in Expression (1), and the unit time is substituted for “time.”

Second Consumed Calories=METs×Time×Weight×1.05 (1)

A calorie difference between the first consumed calories and the second consumed calories may be obtained, and the calorie difference may be converted into METs in accordance with Expression (2) indicated below, The same values as in Expression (1) may be substituted for “time” and “weight” in Expression (2).

METs=(First Consumed Calories−Second Consumed Calories)/(Time×Weight×1.05) (2)

In accordance with Expression (3) indicated below, the correction value may be calculated from the converted METs. In an embodiment, the correction coefficient in Expression (3) may be a value (e.g., “6”) obtained through experiment conducted by the inventor or the like. In another embodiment, the correction coefficient may be varied depending on the user's height, weight, age, sex, or the like.

Correction Value=Difference in Exercise Intensity (METs)×Correction Coefficient (3)

When the correction value is calculated, the corrected maximum heart rate including a value obtained by adding the correction value to the current maximum heart rate may be registered again as one piece of user information. A maximum-heart-rate table corresponding to the corrected maximum heart rate may be set again. For example, when the correction value is “10” and the current maximum heart rate is “180”, the corrected maximum heart rate may be “190”. A maximum-heart-rate table corresponding to 190 bpm may be set again.

When the cardiopulmonary function has been changed and the current and past heart rates with the same training differ from each other, the maximum heart rate can be corrected depending on the change in user's cardiopulmonary function utilizing the difference in exercise intensity when the same training is performed. Since the user's maximum heart rate is corrected, the training performed by the user can be managed appropriately.

For example, since the maximum-heart-rate table will be set again if the user's maximum heart rate is corrected, consumed calories when the training is performed can be obtained properly. Since the exercise intensity obtained using the maximum heart rate becomes a proper value, the user can appropriately perform a training which produces an effect that he/she expects.

In an embodiment, by estimating the difference in exercise intensity, the maximum heart rate can be corrected. By using METs for estimating the difference in exercise intensity, the correction value can be obtained by calculation.

In another embodiment, after correcting the maximum heart rate, it may be verified whether the correction is proper. For example, when a training is performed after the correction, the first consumed calories and the second consumed calories may be calculated, and the calorie difference may be calculated again. When the calorie difference exceeds a predetermined value, the maximum heart rate may be corrected again assuming that the maximum heart rate has not been corrected properly. In still another embodiment, a numerical value, such as the maximum oxygen uptake, may be obtained to calculate the maximum heart rate separately, and it may be determined whether the correction has been performed properly depending on whether the separately calculated maximum heart rate corresponds or substantially corresponds with the corrected maximum heart rate.

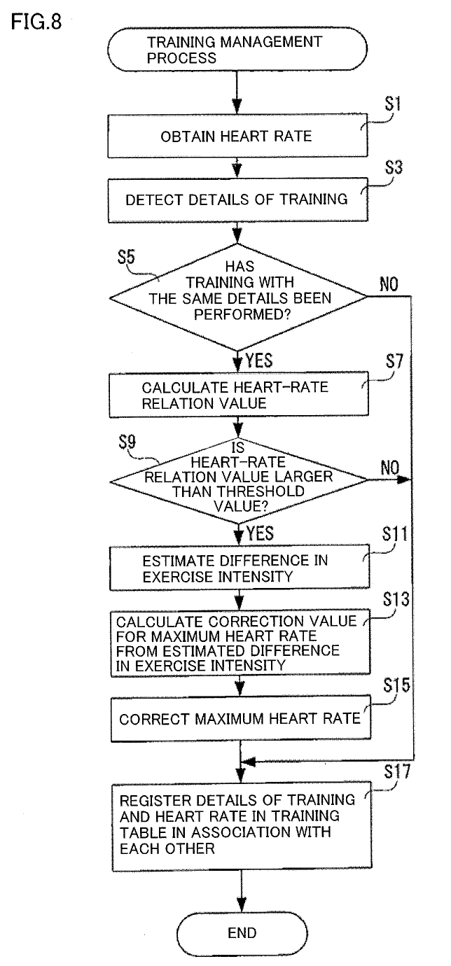

The characteristics of an embodiment have been described above. They will be described in detail using the memory map shown in FIG. 7 and a flowchart shown in FIG. 8.

Referring to FIG. 7, RAM 46 can include a program storage area 302 and a data storage area 304. Program storage area 302 includes an area in which part or whole of program data preset in flash memory 44 (FIG. 2) is read and stored (developed), as described previously.

Program storage area 302 can store a training management program 310 for correcting the user's maximum heart rate and registering the history of trainings, and the like, Program storage area 302 can also store a program for executing a music player function and the like.

An angular velocity buffer 330, an acceleration buffer 332, an atmospheric pressure buffer 334, a heart rate buffer 336, and the like may be ensured in data storage area 304 of RAM 46. Data storage area 304 can store user information data 338, maximum-heart-rate data 340, METs table 342, training table 344, and the like.

Angular velocity buffer 330 can temporarily store each of the triaxial angular velocities output from attitude sensor 54. Acceleration buffer 332 can temporarily store each of the triaxial accelerations output from attitude sensor 54. Atmospheric pressure buffer 334 can temporarily store the surrounding atmospheric pressure detected by atmospheric pressure sensor 56. Heart rate buffer 336 can temporarily store the heart rate measured by heart rate sensor 78 and received from headphone 12.

User information data 338 includes information, such as the height, weight, age, sex, maximum heart rate, resting heart rate, and the like of a user. Maximum-heart-rate data 340 includes data including a plurality of maximum-heart-rate tables shown in FIG. 6, for example. METs table 342 includes a table storing METs corresponding to the speed at which the user is moving and the inclination of the road on which the user is moving. Training table 344 may store detected training information which is a history of trainings performed by the user.

Data storage area 304 may temporarily store map data to be displayed when a training is performed, and may store address book data and another flag or timer (counter) necessary for executing a program.

Processor 30 can process in parallel a plurality of tasks including the training management process shown in FIG. 8 and the like under control of a Windows® OS (Operating System) or another OS, such as a Linux® OS including Android™ and iOS®.

FIG. 8 shows an example of a flowchart of a training management process. For example, a user executes the training controlling function to perform an operation for starting a training, the training management process may be started. When the training controlling function is executed, mobile phone 10 can issue an instruction to headphone 12 to transmit data indicating a heart rate. Headphone 12 can measure the heart rate in response to this instruction, and can transmit the heart rate to mobile phone 10.

In step S1, processor 30 can obtain a heart rate. The user's current heart rate can be read from heart rate buffer 336. In step S3, processor 30 can detect the details of a training. The details of a training being currently performed by the user can be analyzed by reading the angular velocities from angular velocity buffer 330, the accelerations from acceleration buffer 332, and the atmospheric pressure from atmospheric pressure buffer 334.

In step S5, processor 30 can determine whether or not a training with the same details has been performed. It can be determined whether a history including a training corresponding with the analyzed training has been registered in training table 344. When “NO” in step S5, that is, when the same training has not been performed, processor 30 proceeds to step S17.

When “YES” in step S5, for example, when the analysis result of the training being currently performed is “running at 9.7 km/h on a road with an inclination of 5%”, and when a history of the same training as the training indicated by this analysis result has been registered in training table 344, processor 30 can calculate a heart-rate relation value in step S7. The difference between the heart rate in the training information registered in training table 344 and the heart rate obtained in step S1 can be calculated.

In step S9, processor 30 can determine whether or not the heart-rate relation value is larger than a threshold value. When the training having been performed and the training being currently performed are the same, it can be determined whether the user's cardiopulmonary function has been changed. When “NO” in step S9, that is, when the user's cardiopulmonary function has not been changed, processor 30 can proceed to step S17.

When “YES” in step S9, for example, when the user's cardiopulmonary function has been changed and the difference in heart rate is larger than the threshold value, the correction value can be calculated by steps S11 and S13 based on the heart-rate relation value. In step S11, processor 30 can estimate the difference in exercise intensity. As described above, the first consumed calories and the second consumed calories can be calculated, and the calorie difference can be converted into METs. In step S13, processor 30 can calculate the correction value for the maximum heart rate from the estimated difference in exercise intensity. The correction value can be calculated from the converted METs in accordance with Expression (3).

In step S15, processor 30 can correct the maximum heart rate. The calculated correction value can be added to the maximum heart rate included in user information data 338 to replace the maximum heart rate included in user information data 338 with the corrected maximum heart rate. The maximum-heart-rate table can be set again in response to the correction of the maximum heart rate.

In step S17, processor 30 can register the details of the training and the heart rate in training table 344 in association with each other. For example, when the analysis result of the training is “running at 9.7 km/h on a road with an inclination of 5%” and the current heart rate is 126 bpm, training information associating them with each other can be created, and the training information can be registered in training table 344. When step S17 is terminated, processor 30 can terminate the training management process.

In another embodiment, the start of a training may be automatically determined using the output (e.g., accelerations) of attitude sensor 54. The training management process may be executed when it is determined that a training has been started if the user performs the training, regardless of a user's operation.

In still another embodiment, the second consumed calories may be converted from METs using a table or the like. The second consumed calories may be calculated using a mathematical expression different from Expression (1).

In still another embodiment, the maximum heart rate may be obtained using an approach other than the Karvonen Formula.

In another embodiment, METs may be obtained using a numerical value different from the speed at which the user is moving and the inclination of the road on which the user is moving.

In still another embodiment, the correction value may be calculated using a mathematical expression different from Expression (3). The correction value may be obtained using another approach different from a mathematical expression.

In still another embodiment, a headset with a microphone added to headphone 12 may be used. Mobile phone 10 and headphone 12 may be wire connected. Headphone 12 may also be called an earphone. Although ear mounted unit 76 of headphone 12 in an embodiment is of a so-called in-ear type, but may be a canal type or a head-mounted type in another embodiment.

In still another embodiment, heart rate sensor 78 may be mounted on a wristwatch type wearable terminal or the like rather than headphone 12, and may be worn on a user's arm. The wearable terminal may include mobile phone 10. The user can easily manage a training merely by wearing the wearable terminal.

In still another embodiment, heart rate sensor 78 for directly measuring the heart rate may be employed. A user wears a chest belt including this heart rate sensor 78. A sensor including a LED for emitting green light may be employed as heart rate sensor 78. As heart rate sensor 78, a MEMS device for detecting vibrations near a blood vessel may be used, or a sensor for detecting changes in movement (pulse) of a blood vessel in accordance with an acquired moving image may be employed.

In another embodiment, the analysis result of a training may indicate the type of the training by an identification number assigned in advance. The analysis result may be such a simple one that lists numerical values of the inclination, speed and identification number.

Although GUI and the like are not illustrated, a user can freely change his/her own user information at any timing.

Although the above embodiment does not describe specifically, the maximum heart rate may be corrected even if the user's cardiopulmonary function degrades. For example, it is assumed that the user's cardiopulmonary function degrades when the user no longer performs a training. The user's maximum heart rate may be corrected to be smaller.

In still another embodiment, the ratio of the current heart rate while a training is being performed to the heart rate in the past when a training was performed or the ratio of the heart rate in the past to the current heart rate may be calculated as the heart-rate relation value. Based on the calculated ratio, the difference in exercise intensity may be estimated, and the correction value may be calculated.

In the above-described embodiment, the word “larger” is used for a threshold value (such as a predetermined value). The expression “larger than a threshold value” also includes the meaning of “larger than or equal to the threshold value.” The expression “smaller than a threshold value” also includes the meaning of “less than or equal to a threshold value” and “less than a threshold value.”

The program used in an embodiment may be stored in a HDD (Hard Disc Drive) of a data distribution server, and may be distributed to mobile phone 10 over a network. A storage medium, such as an optical disk including a CD (Compact Disc), a DVD (Digital Versatile Disc) and a BD (Blu-Ray Disc), a USB (Universal Serial Bus) memory, and a memory card, with a plurality of programs stored therein, may be sold or distributed. When a program downloaded through the above-described server, storage medium or the like is installed in a mobile phone having a configuration equivalent to that of an embodiment, effects equivalent to those of an embodiment are obtained.

All of specific numerical values mentioned in the present specification are mere examples, and can be modified as appropriate depending on changes in product specifications or the like.

Although the present disclosure has been described and illustrated in detail, it is clearly understood that the same is by way of illustration and example only and is not to be taken by way of limitation, the scope of the present disclosure being interpreted by the terms of the appended claims.

1. A mobile terminal for managing a training performed by a user based on a predetermined maximum heart rate, the mobile terminal comprising:
a memory configured to store details of a training having been performed and a heart rate in the training in association with each other; and
at least one processor,
the at least one processor being configured to:
obtain a heart rate of the user, an attitude of the mobile terminal and an atmospheric pressure surrounding the mobile terminal;
while a first training is being performed, detect details of the first training from the attitude and the atmospheric pressure;
determine whether a training with details corresponding with the details of the first training has been performed; and
when the details of the first training correspond with details of a second training having been performed, correct the maximum heart rate based on a first heart rate associated with the first training and a second heart rate associated with the second training.

2. The mobile terminal according to claim 1, wherein the at least one processor is configured to:
if the first training has been performed, calculate a heart-rate relation value from the first heart rate and the second heart rate;
calculate a correction value for correcting the maximum heart rate based on the heart-rate relation value; and
correct the maximum heart rate based on the correction value.

3. The mobile terminal according to claim 2, wherein
the heart-rate relation value includes a difference between the second heart rate and the first heart rate, and
the at least one processor is configured to:
calculate the difference between the second heart rate and the first heart rate; and
when the difference is larger than a threshold value, calculate the correction value for correcting the maximum heart rate.

4. The mobile terminal according to claim 2, wherein the at least one processor is configured to:
estimate a difference in exercise intensity between an exercise intensity when the second training is performed and an exercise intensity when the first training is performed, based on the heart-rate relation value; and
calculate the correction value based on the difference in exercise intensity.

5. The mobile terminal according to claim 1, wherein the at least one processor is configured to store in the memory the details of the first training and the first heart rate in association with each other.

6. The mobile terminal according to claim 1, further comprising a headphone including a heart-rate measuring sensor, wherein
the at least one processor is configured to obtain a heart rate measured by the heart-rate measuring sensor.

7. A processor-readable non-transitory recording medium with a program recorded thereon, the program causing a processor of a mobile terminal to execute a training management method for managing a training performed by a user based on a predetermined maximum heart rate,
the training management method comprising:
obtaining a heart rate of the user, an attitude of the mobile terminal and an atmospheric pressure surrounding the mobile terminal;
while a first training is being performed, detecting details of the first training from the attitude and the atmospheric pressure;
determining whether a training with details corresponding with the details of the first training has been performed; and
when the details of the first training correspond with details of a second training having been performed, correcting the maximum heart rate based on a first heart rate associated with the first training and a second heart rate associated with the second training.

8. A training management method for managing a training performed by a user based on a predetermined maximum heart rate, the training management method being executed by a processor of a mobile terminal,
the training management method comprising:
obtaining a heart rate of the user, an attitude of the mobile terminal and an atmospheric pressure surrounding the mobile terminal;
while a first training is being performed, detecting details of the first training from the attitude and the atmospheric pressure;
determining whether a training with details corresponding with the details of the first training has been performed; and
when the details of the first training correspond with details of a second training having been performed, correcting the maximum heart rate based on a first heart rate associated with the first training and a second heart rate associated with the second training.
